(19) United States
(12) Patent Application Publication
(10) Pub. No.: US 2004/0235863 A1
(43) Pub. Date: Nov. 25, 2004

(54) Morpholine-bridged pyrazolopyridine derivatives

(76) Inventors: Achim Feurer, Wilhelmsfeld (DE); Dietmar Flubacher, Freiburg (DE); Stefan Weigand, Wuppertal (DE); Johannes-Peter Stasch, Solingen (DE); Elke Stahl, Bergisch Gladbach (DE); Thomas Schenke, Bergisch Gladbach (DE); Cristina Alonso-Alija, Haan (DE); Frank Wunder, Wuppertal (DE); Dieter Lang, Velbert (DE); Klaus Dembowsky, Munchen (DE); Alexander Straub, Wuppertal (DE); Elisabeth Perzborn, Wuppertal (DE)

Correspondence Address: Jeffrey M Greenman, Bayer Corporation, 400 Morgan Lane, West Haven, CT, 06516, US

(21) Appl. No.: 10/482,766

(22) PCT Filed: Jun. 25, 2002
(86) PCT No.: PCT/EP02/06991

(30) Foreign Application Priority Data
Jul. 4, 2001 (DE) 101 32 416.2

(51) Int. Cl. A61K 31/506; C07D 471/02

(52) U.S. Cl. 514/256; 544/328

(57) ABSTRACT

The invention relates to novel pyrazolopyridine derivatives of formula (I), wherein R¹ represents (a) or (b) and n represents 1 or 2 and R² represents H NH₂. The invention also relates to salts, isomers and hydrates thereof as stimulators for soluble guanylate cyclase and to their use as agents in the treatment of cardiovascular diseases, hypertonicity, thrombo-embolic diseases and ischemia, sexual dysfunction or inflammations and for the treatment of diseases of the central nervous system.

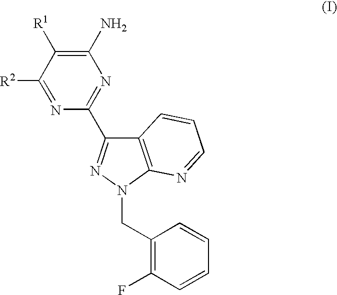

The present invention relates to novel chemical compounds which stimulate soluble guanylate cyclase, to the preparation thereof and to the use thereof as medicaments, in particular as medicaments for the treatment of cardiovascular disorders.

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triposphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. The latter is of central importance for the mechanism of activation. NO is able to bind to the iron atom of heme and thus markedly increase the activity of the enzyme. Heme-free preparations cannot, by contrast, be stimulated by NO. CO is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes. Under pathophysiological conditions, the NO/cGMP system may be suppressed, which may lead for example to high blood pressure, platelet activation, increased cellular proliferation, endothelial dysfunction, atherosclerosis, angina pectoris, heart failure, thromboses, stroke and myocardial infarction.

A possible way of treating such disorders which is independent of NO and aims at influencing the cGMP signal pathway in organisms is a promising approach because of the high efficiency and few side effects which are to be expected.

Compounds, such as organic nitrates, whose effect is based on NO have to date been exclusively used for the therapeutic stimulation of soluble guanylate cyclase. NO is produced by bioconversion and activates soluble guanylate cyclase by attaching to the central iron atom of heme. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole (YC-1, Wu et al., Blood 84 (1994), 4226; Mülsch et al., Br. J. Pharmacol. 120 (1997), 681), fatty acids (Goldberg et al, J. Biol. Chem. 252 (1977), 1279), diphenyliodonium hexafluorophosphate (Pettibone et al., Eur. J. Pharmacol. 116 (1985), 307), isoliquiritigenin (Yu et al., Brit. J. Pharmacol. 114 (1995), 1587) and various substituted pyrazole derivatives (WO 98/16223).

In addition, WO 98/16507, WO 98/23619, WO 00/06567, WO 00/06568, WO 00/06569 and WO 00/21954 describe pyrazolopyridine derivatives as stimulators of soluble guanylate cyclase. Also described in these patent applications are pyrazolopyridines having a pyrimidine residue in position 3. Compounds of this type have very high in vitro activity in relation to stimulating soluble guanylate cyclase. However, it has emerged that these compounds have some disadvantages in respect of their in vivo properties such as, for example, their behavior in the liver, their pharmacokinetic behavior, their dose-response relation or their metabolic pathway.

It was therefore the object of the present invention to provide further pyrazolopyridine derivatives which act as stimulators of soluble guanylate cyclase but do not have the disadvantages, detailed above, of the compounds from the prior art.

This object is achieved according to the present invention through the compounds as claimed in claim 1. These novel pyrazolopyridine derivatives are distinguished by having in position 3 a pyrimidine residue which has a particular substitution pattern, namely a bridged morpholine residue in position 5 of the pyrimidine ring, and one or two amino groups in position 4 or position 4,6 of the pyrimidine ring.

Specifically, the present invention relates to the compounds of the formula (I)

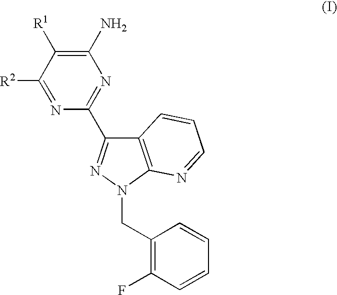

in which

R¹ is

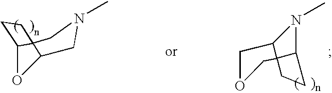

in which

n is 1 or 2;

R² is H or NH₂;

and salts, isomers and hydrates thereof.

In an alternative embodiment, the present invention relates to compounds of the formula (I) in which

R¹ is

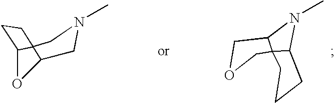

R² is H or NH₂;

and salts, isomers and hydrates thereof

In a further alternative embodiment, the present invention relates to compounds of the formula (I) in which

R¹ is

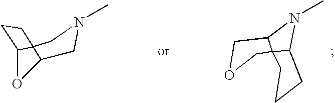

R² is H;

and salts, isomers and hydrates thereof.

The compounds of the invention of the formula (I) may also be in the form of their salts. Mention may generally be made here of salts with organic or inorganic bases or acids.

Physiologically acceptable salts are preferred for the purposes of the present invention. Physiologically acceptable salts of the compound according to the invention may be salts of the substances according to the invention with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, p-toluenesulfonic acid, benzenesulfonic acid, naphthalenedisulfonic acid, acetic acid, propionic acid, lactic acid, tartaric acid, citric acid, fumaric acid, maleic acid or benzoic acid.

Physiologically acceptable salts may likewise be metal or ammonium salts of the compound according to the invention having a free carboxyl group. Particularly preferred examples are sodium, potassium, magnesium or calcium salts, and ammonium salts derived from ammonia or organic amines such as, for example, ethylamine, di- or triethylamine, di- or triethanolamine, dicyclohexylamine, dimethylaminoethanol, arginine, lysine or ethylenediamine.

The compounds of the invention may exist in tautomeric forms. This is known to the skilled worker, and such forms are likewise encompassed by the invention.

The compounds of the invention may additionally occur in the form of their possible hydrates.

The compounds of the formula (I) of the invention can be prepared by reacting the compound of the formula (II)

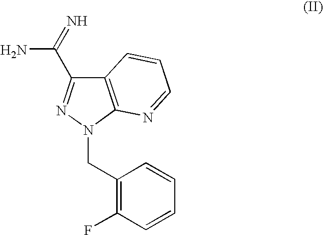

A) with a compound of the formula (III)

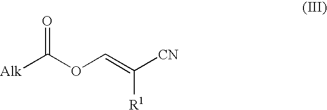

where

R¹ is as defined above;

Alk is linear or branched C₁₋₄-alkyl;

where appropriate in an organic solvent with heating to give the compound of the formula (I);

or

B) with a compound of the formula (IV)

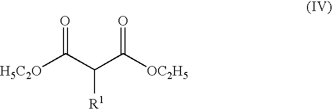

where

R¹ is as defined above;

in an organic solvent with heating to give compounds of the formula (V)

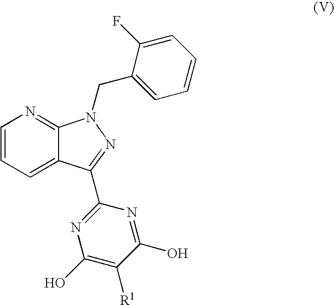

where

R¹ is as defined above;

subsequently with a halogenating agent to give compounds of the formula (VI)

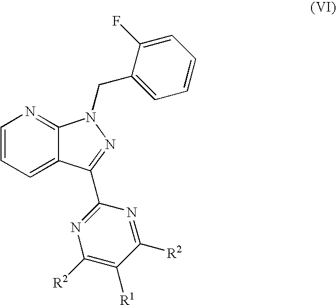

where

R¹ is as defined above;

R² is halogen;

and finally with aqueous ammonia solution with heating under elevated pressure.

The compound of the formula (II) can be prepared as shown in the following reaction scheme:

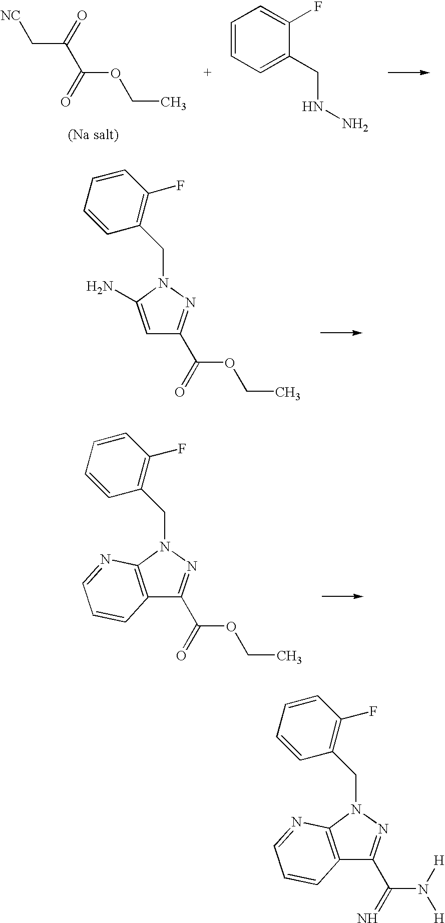

The compound of the formula (II) can be obtained in a multistage synthesis from the sodium salt of ethyl cyanopyruvate, which is known from the literature (Borsche and Manteuffel, Liebigs. Ann. Chem. 1934, 512, 97). Reaction thereof with 2-fluorobenzylhydrazine with heating under a protective gas atmosphere in.an inert solvent such as dioxane results in ethyl 5-amino-1-(2-fluorobenzyl)pyrazole-3-carboxylate, which cyclizes to give the corresponding pyridine derivative by reaction with dimethylaminoacrolein with heating in an acidic medium under a protective gas atmosphere. This pyridine derivative ethyl 1-(2-fluorobenzyl)-1H-pyrazolo[3,4-b]pyridine-3-carboxylate is converted by a multistage sequence consisting of conversion of the ester with ammonia into the corresponding amide, dehydration with a dehydrating agent such as trifluoroacetic anhydride to give the corresponding nitrile derivative, reaction of the nitrile derivative with sodium ethoxide and final reaction with ammonium chloride into the compound of the formula (II).

The compounds of the formula (III) can be prepared for example as shown in the following schemes:

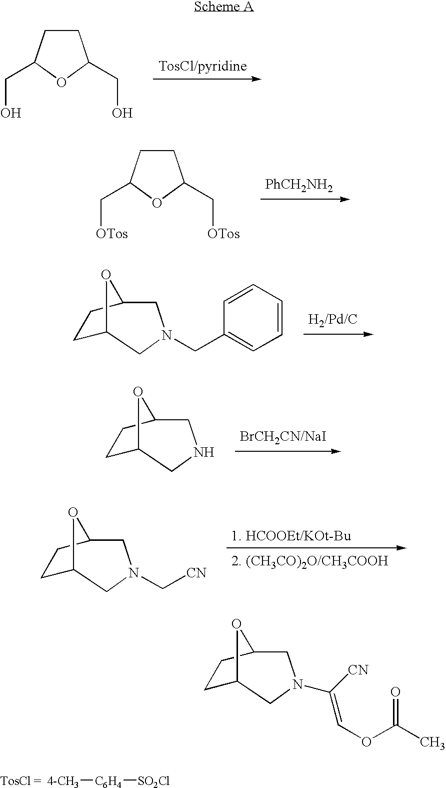

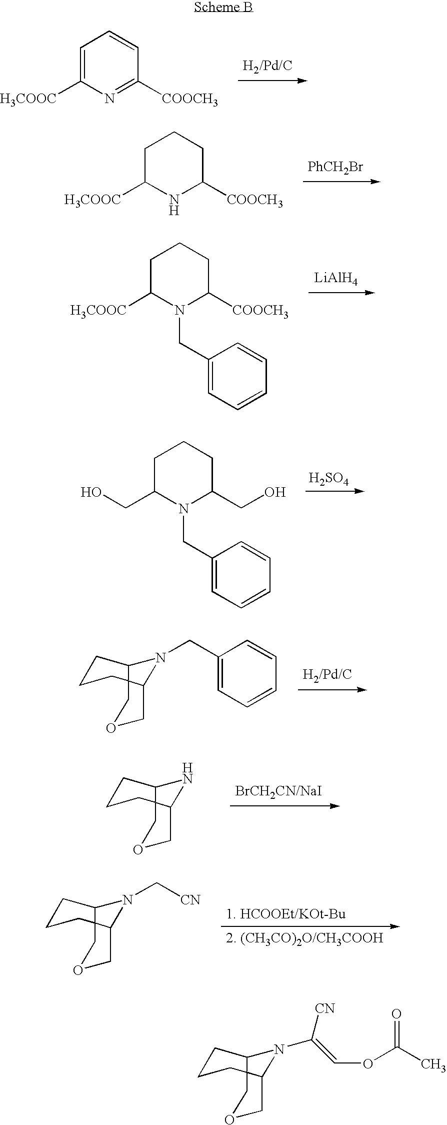

The corresponding starting compounds 2,5-bis(hydroxymethyl)tetrahydrofuran and dimethyl pyridine-2,6-dicarboxylate can be purchased (e.g. from Aldrich) or can be obtained in a conventional manner by routes known to the skilled worker.

In the case of the bicyclic [3.2.1]octane, the bicyclic system is assembled for example by reacting the bishydroxymethyltetrahydrofuran derivative (activated as bistosylate) with benzylamine in a nucleophilic substitution reaction under conditions conventionally used for such reactions. It is preferred according to the invention to carry out the reaction in an organic solvent, for example a hydrocarbon, preferably an aromatic hydrocarbon, and especially toluene, with use of a 2-5-fold excess of the amine, preferably under atmospheric pressure and stirring the reaction solution for a plurality of hours, for example 2 hours, at elevated temperature, for example 60-130° C., preferably 80-120° C., in particular 100° C.

In the case of the bicyclic [3.3.1]nonane, the bicyclic system is assembled for example by an intramolecular nucleophilic substitution reaction of the two hydroxyl groups of the piperidine 2,6-dihydroxymethyl derivative under conditions conventionally used for such reactions. It is preferred according to the invention to carry out the reaction under acidic conditions, for example in the presence of concentrated sulfuric acid, preferably under atmospheric pressure and stirring the reaction solution for a plurality of hours, for example 24 hours, at elevated temperature, for example 60-200° C., preferably 80-190° C., in particular 175° C. The piperidine 2,6-dihydroxymethyl derivative required for this can be prepared from pyridine-2,6-dicarboxylic acid methyl ester by hydrogenation under conditions conventionally used for such reactions, for example with hydrogen on a palladium/activated carbon catalyst, to give the corresponding piperidine-2,6-dicarboxylic acid methyl ester, benzylation of the ring nitrogen with, for example, benzyl bromide (cf. Goldspink, Nicholas J.,: Simpkins, Nigel S.; Beckmann, Marion; Syn. Lett.; 8; 1999; 1292-1294) and subsequent reduction of the carboxylic ester groups to the corresponding hydroxymethyl radicals under conditions conventionally used for such reactions, for example with lithium aluminum hydride in an organic solvent, for example an ether, preferably diethyl ether, using a 2-5-fold excess of the reducing agent, preferably under atmospheric pressure and stirring the reaction solution for a plurality of hours, for example 3 hours, at elevated temperature, for example 30-100° C., preferably 30-70° C., in particular under reflux of the solvent used.

The bicyclic system obtained in this way can in each case be converted with elimination of the benzylic protective group under conditions conventionally used for such reactions, for example with hydrogen on a palladium/activated carbon catalyst in an organic solvent, for example an alcohol, preferably ethanol, preferably under elevated pressure of 50-200 bar, preferably 100 bar, and stirring the reaction solution for a plurality of hours, for example 5 hours, at elevated temperature, for example 60-130° C., preferably 80-120° C., in particular 100° C., into the corresponding bicyclic amines. The latter can be converted by reacting with suitable acetonitrile derivatives, for example with haloacetonitriles and preferably with bromoacetonitrile, under conditions conventionally used for such reactions, for example in an organic solvent such as N,N-dimethylformamide (DMF), using a slight excess of the acetonitrile derivative in the presence of a base, for example an amine such as N,N-diisopropylethylamine, and a halide such as sodium iodide, preferably under atmospheric pressure and stirring the reaction solution for a plurality of hours, for example 24 hours, at elevated temperature, for example 40-130° C., preferably 40-100° C., in particular 60° C., into the corresponding N-methylnitrile derivatives. The compounds of the formula (III) can finally be prepared from the latter by reaction with a formic ester such as, for example, ethyl formate under conditions conventionally used for such reactions, for example in an organic solvent, for example an ether, preferably a cyclic ether such as tetrahydrofuran (THF), using a 2-5-fold excess of formic ester, preferably under atmospheric pressure and stirring the reaction solution for a plurality of minutes, for example 20-60 minutes, at room temperature, and subsequent acetylation with acetic anhydride in the presence of acetic acid under conditions conventionally used for such reactions, for example using a slight excess of acetic anhydride, preferably under atmospheric pressure and stirring the reaction solution for a plurality of minutes, for example 20-60 minutes.

Reaction of compounds of the formulae (II) and (III) to give compounds of the formula (I) can be carried out by using the reactants in equimolar amounts or by using the compound of the formula (III) in slight excess in an organic solvent, for example a hydrocarbon, preferably an aromatic hydrocarbon and in particular toluene, preferably under atmospheric pressure and stirring the reaction solution for a plurality of hours, for example 12 hours, at elevated temperature, for example 80-160° C., preferably 100-150° C., in particular 120° C.

The compounds of the formula (IV) can be obtained commercially (e.g. from Mercachem) or can be prepared in a manner known to the skilled worker.

Reaction of compounds of the formulae (II) and (IV) to give compounds of the formula (V) can be carried out by using the reactants in equimolar amounts or by using the compound of the formula (IV) in slight excess in an organic solvent, for example a hydrocarbon, preferably an aromatic hydrocarbon and in particular toluene, preferably under atmospheric pressure and stirring the reaction solution for a plurality of hours, for example 12 hours, at elevated temperature, for example 80-160° C., preferably 100-150° C., in particular 140° C.

Reaction of compounds of the formula (V) to give compounds of the formula (VI) can be carried out by reacting the compounds of the formula (V) with a halogenating agent, where appropriate in an organic solvent conventionally used for reactions of this type, such as; for example, dimethylformamide (DMF), preferably under atmospheric pressure and stirring the reaction solution for a plurality of hours, preferably 3 hours, at elevated temperature, for example 80-160° C., preferably 100-120° C. POCl₃ can be employed as halogenating agent with preference according to the invention.

Reaction of compounds of the formula (VI) to give the compounds of the invention of the formula (I) can be carried out by reacting the compounds of the formula (VI) with aqueous ammonia solution, preferably under elevated pressure, for example by the reaction taking place in an autoclave, so that the reaction proceeds under the autogenous pressure of the reaction mixture, and stirring the reaction solution for a plurality of hours, for example 12 hours, at elevated temperature, for example 80-160° C., preferably 100-150° C., in particular 140° C.

The compounds of the invention of the formula (I) shows a valuable range of pharmacological effects which could not have been predicted.

The compounds of the invention of the formula (I) lead to vasorelaxation, inhibition of platelet aggregation and to a reduction in blood pressure and to an increase in coronary blood flow. These effects are mediated by direct stimulation of soluble guanylate cyclase and an intracellular increase in cGMP. In addition, the compound of the invention of the formula (I) enhances the effect of substances which increase the cGMP level, such as, for example, EDRF (endothelium derived relaxing factor), NO donors, protoporphyrin IX, arachidonic acid or phenylhydrazine derivatives.

They can therefore be employed in medicaments for the treatment of cardiovascular disorders such as, for example, for the treatment of high blood pressure and heart failure, stable and unstable angina pectoris, peripheral and cardiac vascular disorders, of arrhythmias, for the treatment of thromboembolic disorders and ischemias such as myocardial infarction, stroke, transistorily and ischemic attacks, disturbances of peripheral blood flow, prevention of restenoses as after thrombolysis therapies, percutaneously transluminal angioplasties. (PTAs), percutaneously transluminal coronary angioplasties (PTCAs), bypass and for the treatment of arteriosclerosis, asthmatic disorders and diseases of the urogenital system such as, for example, prostate hypertrophy, erectile dysfunction, female sexual dysfunction, osteoporosis, gastroparesis and incontinence.

The compounds of the formula (I) described in the present invention are also suitable as active ingredients for controlling central nervous system diseases characterized by disturbances of the NO/cGMP system. They are suitable in particular for improving perception, concentration, learning, or memory after cognitive impairments like those occurring in particular in situations/disorders/syndromes such as mild cognitive impairment, age-associated learning and memory impairments, age-associated memory losses, vascular dementia, craniocerebral trauma, stroke, dementia occurring after strokes (“post stroke dementia”), post-traumatic craniocerebral trauma, general concentration impairments, concentration impairments in children with learning and memory problems, Alzheimer's disease, vascular dementia, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob dementia, HIV dementia, schizophrenia with dementia or Korsakoff's psychosis. They are also suitable for the treatment of disorders of the central nervous system such as states of anxiety, tension and depression, CNS-related sexual dysfunctions and sleep disturbances, and for controlling pathological disturbances of the intake of food, stimulants and addictive substances.

The active ingredients are furthermore also suitable for controlling cerebral blood flow and thus represent effective agents for controlling migraine.

They are also suitable for the prophylaxis and control of the sequelae of cerebral infarctions such as stroke, cerebral ischemias and craniocerebral trauma. The compounds of the invention of the formula (I) can likewise be employed for controlling states of pain.

In addition, the compounds of the invention have an antiinflammatory effect and can therefore be employed as antiinflammatory agents.

Furthermore the invention encompasses the combination of the compounds of the invention of the formula (I) with organic nitrates and NO donors.

Organic nitrates and NO donors for the purposes of the invention are generally substances which display their therapeutic effect via release of NO or NO species. Preference is given to sodium nitroprusside, nitroglycerine, isosorbide dinitrate, isosorbide mononitrate, molsidomine and SIN-1.

In addition, the invention encompasses the combination with compounds which inhibit the breakdown of cyclic guanosine monophosphate (cGMP). These are in particular inhibitors of phosphodiesterases 1, 2 and 5; nomenclature of Beavo and Reifsnyder (1990), TiPS 11 pp. 150 to 155. These inhibitors potentiate the effect of the compounds of the invention, and the desired pharmacological effect is increased.

Biological Investigations

Vasorelaxant Effect in Vitro

Rabbits are stunned by a blow to the back of the neck and are exsanguinated. The aorta is removed, freed of adherent tissue, divided into rings 1.5 mm wide and put singly under tension in 5 ml organ baths containing carbogen-gassed Krebs-Henseleit solution at 37° C. with the following composition (mM): NaCl: 119; KCl: 4.8; CaCl₂×2H₂O: 1; MgSO₄×7H₂O: 1.4; KH₂PO₄: 1.2; NaHCO₃: 25; glucose: 10. The force of contraction is detected with Statham UC2 cells, amplified and digitized via A/D converters (DAS-1802 HC, Keithley Instruments Munich) and recorded in parallel on chart recorders. A contraction is generated by adding phenylephrine to the bath cumulatively in increasing concentration. After several control cycles, the substance to be investigated is investigated in each further run in increasing dosage in each case, and the height of the contraction is compared with the height of the contraction reached in the last preceding run. The concentration necessary to reduce the height of the control value by 50% (IC₅₀) is calculated from this. The standard application volume is 5 μl, and the DMSO content in the bath solution corresponds to 0.1%. The results are listed in Table 1 below:

TABLE 1
Vasorelaxant effect in vitro

| Example No. | IC₅₀ [μM] |
|---|---|
| 1 | 0.27 |
| 2 | 0.30 |

Determination of the Liver Clearance in vitro

Rats are anesthetized and heparinized, and the liver is perfused in situ through the portal vein. Primary rat hepatocytes are then obtained ex vivo from the liver using collagenase solution. 2-10⁶ hepatocytes per ml were in each case incubated with the same concentration of the compound to be investigated at 37° C. The decrease in the substrate to be investigated over time was determined bioanalytically (HPLC/UV, HPLC/fluorescence or LC/MSMS) at in each case 5 timepoints in the period 0-15 min after the start of incubation. The clearance was calculated therefrom via the number of cells and weight of the liver.

Determination of the Plasma Clearance in vivo

The substance to be investigated is administered intravenously as solution to rats via the tail vein. Blood is taken from the rats at fixed times and is heparinized, and plasma is obtained therefrom by conventional procedures. The substance is quantified in the plasma bioanalytically. The pharmacokinetic parameters are calculated from the plasma concentration/time courses found in this way by conventional non-compartmental methods used for this purpose.

The present invention includes pharmaceutical preparations which, besides non-toxic, inert pharmaceutically suitable carriers, comprise the compound of the invention of the formula (I), and process for the production of these preparations.

The active ingredient may also be present in microencapsulated form in one or more of the carriers indicated above.

The therapeutically effective compound of the formula (I) should be present in the abovementioned pharmaceutical preparations in a concentration of about 0.1 to 99.5, preferably of about 0.5 to 95, % by weight of the complete mixture.

The abovementioned pharmaceutical preparations may, apart from the compound of the invention of the formula (I), also comprise other active pharmaceutical ingredients.

It has generally proved advantageous both in human and in veterinary medicine to administer the active ingredient of the invention in total amounts of about 0.01 to about 700, preferably 0.01 to 100, mg/kg of body weight per 24 hours, where appropriate in the form of a plurality of single doses, to achieve the desired results. A single dose comprises the active ingredient of the invention preferably in amounts of about 0.1 to about 80, in particular 0.1 to 30, mg/kg of body weight.

The present invention is described in detail below by means of non-restrictive preferred examples. Unless indicated elsewhere, all quantitative data relate to percentages by weight.

### EXAMPLES

Abbreviations:

RT: room temperature

EA: ethyl acetate

MCPBA: m-chloroperoxybenzoic acid

BABA: n-butyl acetate/n-butanol/glacial acetic acid/phosphate buffer pH 6 (50:9:25.15; org. phase)

DMF: N,N-dimethylformamide

Mobile Phases for Thin-Layer Chromatography:

T1 E1: toluene—ethyl acetate (1:1)

T1 EtOH1: toluene—methanol (1:1)

C1 E1: cyclohexane—ethyl acetate (1:1)

C1 E2: cyclohexane—ethyl acetate (1:2)

Methods for Determining the HPLC Retention Times and Preparative Separation Methods:

Method A (HPLC-MS):

Eluent: A=CH₃CN B=0.6 g 30% HCl/1 H₂O

Flow rate: 0.6 ml/min

Column oven: 50° C.

Column: Symmetry C18 2.1 *150 mm

Gradient:

| Time (min) | % A | % B | Flow rate (ml/min) |
|---|---|---|---|
| 0 | 10 | 90 | 0.6 |
| 4 | 90 | 10 | 0.6 |
| 9 | 90 | 10 | 0.8 |

Method B (HPLC):

Eluent: A=5 ml HClO₄/1 H₂O, B=CH₃CN

Flow rate: 0.75 ml/min

L-R temperature: 30.00° C. 29.99° C.

Column: Kromasil C18 60*2 mm

Gradient:

| Time (min) | % A | % B |
|---|---|---|
| 0.50 | 98 | 2 |
| 4.50 | 10 | 90 |
| 6.50 | 10 | 90 |
| 6.70 | 98 | 2 |
| 7.50 | 98 | 2 |

Method C (HPLC):

Eluent: A=H₃PO₄0.01 mol/1, B=CH₃CN

Flow rate: 0.75 ml/min

L-R temperature: 30.01° C. 29.98° C.

Column: Kromasil C18 60*2 mm

Gradient:

| Time (min) | % A | % B |
|---|---|---|
| 0.00 | 90 | 10 |
| 0.50 | 90 | 10 |
| 4.50 | 10 | 90 |
| 8.00 | 10 | 90 |
| 8.50 | 90 | 10 |
| 10.00 | 90 | 10 |

Method D (Chiral HPLC):

Eluent: 50% isohexane, 50% ethanol

Flow rate: 1.00 ml/min

Temperature: 40° C.

Column: 250*4.6 mm, packed with Chiralcel OD, 10 μm

Method E (HPLC-MS):

Eluent: A=CH₃CN B=0.3 g 30% HCl/1 H₂O

Flow rate: 0.9 ml/min

Column oven: 50° C.

Column: Symmetry C18 2.1*150 mm

Gradient:

| Time (min) | % A | % B | Flow rate (ml/min) |
|---|---|---|---|
| 0 | 10 | 90 | 0.9 |
| 3 | 90 | 10 | 1.2 |
| 6 | 90 | 10 | 1.2 |

Method F (Preparative HPLC):

Eluent: A=Milli-Q-water, B=acetonitrile, C=1% trifluoroacetic acid

Flow rate: 25 ml/min

Temperature: 50° C.

Packing material: Kromasil 100 C 18 5 μm 250×20 mm No. 1011314R

Gradient:

| Time (min) | A | B | C |
|---|---|---|---|
| 0 | 72 | 10 | 18 |
| 30 | 32 | 60 | 8 |
| 30.1 | 4 | 95 | 1 |
| 40 | 4 | 95 | 1 |
| 48 | 72 | 10 | 18 |

Method G=(LC-MS):

Eluent: A=acetonitrile+0.1% formic acid

B=water+0.1% formic acid

Flow rate: 25 ml/min

Temperature: 40° C.

Packing material: Symmetry C 18, 50×2.1 mm, 3.5 μm

Gradient:

| Time (min) | A | B |
|---|---|---|
| 0 | 10 | 90 |
| 4.0 | 90 | 10 |
| 6.0 | 90 | 10 |
| 6.1 | 10 | 90 |
| 7.5 | 10 | 90 |

Method I (Preparative HPLC):

Eluent: A=Milli-Q-water+0.6 g of concentrated hydrochloric acid per 1l H2O,

B=acetonitrile

Flow rate: 50 ml/min

Temperature: room temperature

Packing material: YMC-Gel ODS-AQS 11 μm 250×30 mm

Gradient:

| Time (min) | A | B |
|---|---|---|
| 0 | 90 | 10 |
| 30 | 90 | 10 |
| 27 | 2 | 98 |
| 34 | 2 | 98 |
| 34.01 | 90 | 10 |
| 38 | 90 | 10 |

Method for Determining the GC Retention Times:

Method H (GC-MS):

Carrier gas: helium

Flow rate: 1.5 ml/min

Initial temperature: 60° C.

Temperature gradient: 14° C./min to 300° C., then 1 min const. 300° C.

Column: HP-5 30 m×320 μm×0.25 μm (film thickness)

Initial time: 2 min

Front injector temp.: 250° C.

Starting Compounds:

### I. Synthesis of (E/Z)-2-cyano-2-(8-oxa-3-azabicyclo[3.2.1]oct-3-yl)ethenyl acetate

### Ia) 2,5-Anhydro-3,4-dideoxy-1,6-bis-O-[(4-methylphenyl)sulfonyl]hexitol

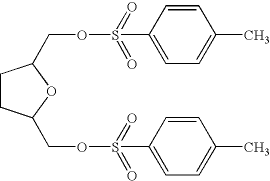

34.0 g (261 mmol) of 2,5-bis(hydroxymethyl)tetrahydrofuran were dissolved in 260 ml of dichloromethane. A solution of 99.0 g (521 mmol) of p-toluenesulfonyl chloride in 52 ml of pyridine and 130 ml of dichloromethane was added dropwise thereto. After stirring at room temperature for 24 hours, the precipitate was filtered off with suction and washed with dichloromethane. The filtrate and the washing phases were combined, washed with dilute hydrochloric acid and subsequently with saturated aqueous sodium bicarbonate solution, dried over magnesium sulfate and evaporated to dryness. The crude product was recrystallized from ethanol.

Yield: 112 g (98%) Melting point: 125° C. MS: (CI pos.), m/z=441 ([M+H]⁺).

### Ib) 3-Benzyl-8-oxa-3-azabicyclo[3.2.1]octane

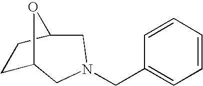

112 g (250 mmol) of 2,5-anhydro-3,4-dideoxy-1,6-bis-O-[(4-methylphenyl)-sulfonyl]hexitol from Ex. Ia and 90.7 g (840 mmol) of benzylamine were heated under reflux in 500 ml of toluene for 20 h. The precipitate was then filtered off with suction and washed with toluene. The combined toluene phases were concentrated in a rotary evaporator and distilled in vacuo. After a fore-run of benzylamine, the product was obtained.

Yield: 28.2 g (54%) Boiling point: 96-99° C. at 8 mbar

MS: (CI pos.), m/z=204 ([M+H]⁺).

### Ic) 8-Oxa-3-azabicyclo[3.2.1]octane hydrochloride

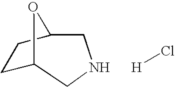

28.2 g (136 mmol) of 3-benzyl-8-oxa-3-azabicyclo[3.2.1]octane from Ex. Ib were dissolved in 200 ml of ethanol and, after addition of 5.00 g of palladium/activated carbon (10%), hydrogenated with 100 bar of hydrogen in an autoclave at 100° C. The catalyst was filtered off with suction and the mother liquor was mixed with 11.9 ml of concentrated hydrochloric acid and concentrated in a rotary evaporator. Acetone was added to the residue, and the resulting precipitate was filtered off with suction and dried over phosphorus pentoxide.

Yield: 17.0 g (84%) Melting point: 209-221° C. MS: (CI pos.), m/z=114 ([M+H]⁺).

### Id 8-Oxa-3-azabicyclo[3.2.1]oct-3-ylacetonitrile

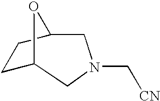

1.54 g (10.3 mmol) of 8-oxa-3-azabicyclo[3.2.1]octane hydrochloride from Ex. Ic were introduced into 20 ml of N,N-dimethylformamide and, while cooling in ice, 2.94 g (22.7 mmol) of N,N-diisopropylethylamine were added. After stirring at room temperature for 30 minutes, 1.36 g (11.4 mmol) of bromoacetonitrile were added dropwise, 89.9 mg (0.60 mmol) of sodium iodide were added, and the mixture was stirred at 60° C. overnight. The reaction mixture was then evaporated to dryness in a rotary evaporator, and the residue was dissolved in a little dichloromethane. The solution was filtered through silica gel with dichloromethane/methanol 50:1 as eluent, and the resulting product fractions were dried under HV.

Yield: 1.24 g (69%) Rf 0.80 (dichloromethane/methanol 20:1) GC-MS: Rₜ=11.23 min (method H). MS (CI pos.), m/z=153 ([M+H]⁺).

### I) (E/Z)-2-Cyano-2-(8-oxa-3-azabicyclo[3.2.1]oct-3-yl)ethenyl acetate

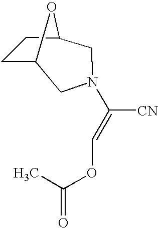

2.00 g (17.8 mmol) of potassium tert-butoxide were introduced into 10 ml of anhydrous tetrahydrofuran. While cooling in ice, a solution of 1.23 g (8.08 mmol of 8-oxa-3-azabicyclo[3.2.1]oct-3-ylacetonitrile from Ex. 1d and 1.37 g (17.8 mmol) of ethyl formate in 5 ml of tetrahydrofuran was added dropwise. After stirring at room temperature for 1 hour, a solution of 1.16 g (11.3 mmol) of acetic anhydride and 1.07 g (17.8 mmol) of acetic acid was added dropwise while cooling in ice, and the mixture was stirred at room temperature for 1 hour. The mixture was subsequently filtered through silica gel with dichloromethane as eluent. The product fractions were evaporated to dryness at 40° C. The product was employed without further purification in the next reaction.

Yield: 2.03 g (54%) Rf. 0.64 (dichloromethane/methanol 20:1)

### II. Synthesis of (E/Z)-2-Cyano-2-(3-oxa-9-azabicyclo[3.3.1]non-9-yl)ethenyl acetate

### IIa) [1-Benzyl-6-(hydroxymethyl)-2-piperidinyl]methanol

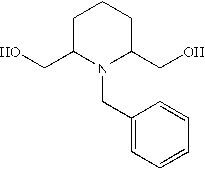

19.0 g (500 mmol) of lithium aluminum hydride were introduced into 300 ml of anhydrous diethyl ether, and a solution of 75.0 g (250 mmol) of dimethyl 1-benzyl-2,6-piperidinedicarboxylate (from dimethyl pyridine-2,6-dicarboxylate by hydrogenation with hydrogen on palladium/activated carbon and subsequent reaction of the dimethyl 2,6-piperidinedicarboxylate formed with benzyl bromide by the method of Goldspink, Nicholas J.; Simpkins, Nigel S.; Beckmann, Marion; Syn. Lett.; 8; 1999; 1292-1294) in 300 ml of anhydrous diethyl ether is added dropwise thereto. The mixture was then heated under reflux for 3 h, cautiously hydrolyzed with 40 ml of water, and mixed with 20 ml of 15% strength aqueous potassium hydroxide solution. The precipitate was filtered off with suction and boiled with dioxane. The combined filtrates were dried over magnesium sulfate and evaporated to dryness in a rotary evaporator. The crude product was subjected to a vacuum distillation.

Yield: 53.3 g (91%) Boiling point: 170° C. at 0.2 mbar

### IIb) 9-Benzyl-3-oxa-9-azabicyclo[3.3.1]nonane

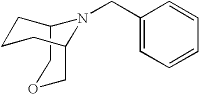

40 g (170 mmol) of [1-benzyl-6-(hydroxymethyl)-2-piperidinyl]methanol from Ex. IIa were stirred in 129 ml of 66% strength sulfric acid at 175° C. overnight. After cooling to room temperature, the mixture was neutralized with sodium carbonate, made alkaline with sodium hydroxide and extracted several times with dichloromethane. The combined organic phases were dried over magnesium sulfate and evaporated to dryness in a rotary evaporator. The residue was distilled in vacuo.

Yield: 26.5 g (72%) Boiling point: 101-103° C. at 8 mbar MS: (CI pos.), m/z=218 ([M+H]⁺).

### IIc) 3-Oxa-9-azabicyclo[3.3.1]nonane hydrochloride

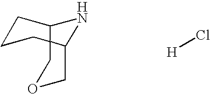

26.0 g (120 mmol) of 9-benzyl-3-oxa-9-azabicyclo[3.3.1]nonane from Ex. IIb were dissolved in 200 ml of ethanol and, after addition of 5.00 g of palladium/activated carbon (10%), hydrogenated with 100 bar of hydrogen in an autoclave at 100° C. The catalyst was filtered off with suction and the mother liquor was mixed with 10.9 ml of concentrated hydrochloric acid and concentrated in a rotary evaporator. Acetone was added to the residue, and the resulting precipitate was filtered off with suction and dried over-phosphorus pentoxide.

Yield: 12.0 g (81%) ¹H NMR: (400 MHz, D₂O), δ=1.68-1.76 (m, 1H, CH), 2.08-2.15 (m, 4H, 2CH₂), 2.32-2.45 (m, 1H, CH), 3.56 (m_{c}, 2H, 2CH), 4.07-4.17 (m, 4H, 2CH₂),

### IId) 3-Oxa-9-azabicyclo[3.3.1]non-9-ylacetonitrile

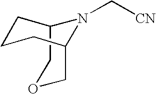

2.00 g (12.2 mmol) of 3-oxa-9-azabicyclo[3.3.1]nonane hydrochloride from Ex. IIc were introduced into 20 ml of N,N-dimethylformamide and, while cooling in ice, 3.10 g (26.9 mmol) of N,N-diisopropylethylamine were added. After stirring at room temperature for 30 minutes, 1.61 g (13.4 mmol) of bromoacetonitrile were added dropwise, 60.0 mg (0.40 mmol) of sodium iodide were added, and the mixture was stirred at 60° C. overnight. The reaction mixture was then evaporated to dryness in a rotary evaporator, and the residue was dissolved in a little dichloromethane. The solution was filtered through silica gel with dichloromethane/methanol 50:1 as eluent, and the resulting product fractions were dried under HV.

Yield: 1.59 g (76%) Rf 0.79 (dichloromethane/methanol 20:1) GC-MS: Rₜ=12:55 min (method H). MS (CI pos.), m/z=167 ([M+H]⁺).

### II) (E/Z)-2-Cyano-2-(3-oxa-9-azabicyclo[3.3.1]non-9-yl)ethenyl acetate

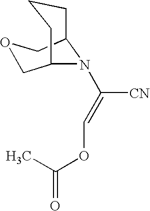

2.35 g (20.9 mmol) of potassium tert-butoxide were introduced into 10 ml of anhydrous tetrahydrofuran. While cooling in ice, a solution of 1.55 g (9.50 mmol) of 3-oxa-9-azabicyclo[3.3.1]non-9-ylacetonitrile from Ex. IId and 1.55 g (20.9 mmol) of ethyl formate in 5 ml of tetrahydrofuran was added dropwise. After stirring at room temperature for 1 hour, a solution of 1.36 g (13.3 mmol) of acetic anhydride and 1.26 g (20.9 mmol) of acetic acid was added dropwise while cooling in ice, and the mixture was stirred at room temperature for 1 hour. The mixture was subsequently filtered through silica gel with dichloromethane as eluent. The product fractions were evaporated to dryness at 40° C. The product was employed without further purification in the next reaction.

Yield: 1.59 g (39%) Rf 0.66 (dichloromethane/methanol 20:1)

### III. Synthesis of 1-(2-fluorobenzyl)1H-pyrazolo[3,4-b]pyridine-3-carboxamidine

### IIIa) Ethyl 5-amino-1-(2-fluorobenzyl)pyrazole-3-carboxylate

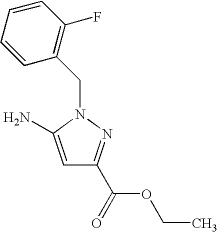

111.75 g (75 ml, 0.98 mol) of trifluoroacetic acid are added to 100 g (0.613 mol) of the sodium salt of ethyl cyanopyruvate (preparation in analogy to Borsche and Manteuffel, Liebigs Ann. 1934, 512, 97) in 2.5 l of dioxane under argon with efficient stirring at room temperature, and the mixture is stirred for 10 min, during which most of the precursor dissolves. Then 85.93 g (0.613 mol) of 2-fluorobenzyl-hydrazine are added, and the mixture is boiled overnight. After cooling, the sodium trifluoroacetate crystals which have separated out are filtered off with suction and washed with dioxane, and the crude solution is reacted further.

### IIIb) Ethyl 1-(2-fluorobenzyl)-1H-pyrazolo[3,4-b]pyridine-3-carboxylate

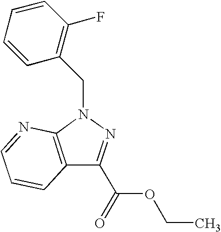

The solution obtained from IIIa) is mixed with 61.25 ml (60.77 g, 0.613 mol) of dimethylaminoacrolein and 56.28 ml (83.88 g, 0.736 mol) of trifluoroacetic acid and boiled under argon for 3 days. The solvent is then evaporated in vacuo, and the residue is added to 2 l of water and extracted three times with 1 l of ethyl acetate each time. The combined organic phases are dried with magnesium sulfate and concentrated in a rotary evaporator. Chromatography is carried out on 2.5 kg of silica gel, eluting with a toluene/toluene-ethyl acetate=4:1 gradient. Yield: 91.6 g (49.9% of theory over two stages).

Melting point 85° C. R_{f} (SiO₂, T1E1): 0.83

### IIIc) 1-(2-Fluorobenzyl)-]H-pyrazolo[3,4-b]pyridine-3-carboxamide

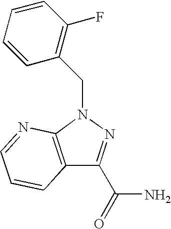

10.18 g (34 mmol) of the ester obtained in example IIIb) are introduced into 150 ml of methanol saturated with ammonia at 0-10° C. Stirring at room temperature for two days is followed by concentration in vacuo.

R_{f} (SiO₂, T1E1): 0.33

### IIId) 3-Cyano-]-(2-fluorobenzyl)-]H-pyrazolo[3,4-b]pyridine

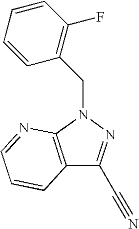

36.1 g (133 mmol) of 1-(2-fluorobenzyl)-1H-pyrazolo[3,4-b]pyridine-3-carboxamide from example IIIc) are dissolved in 330 ml of THF, and 27 g (341 mmol) of pyridine are added. Then, over the course of 10 min, 47.76 ml (71.66 g, 341 mmol) of trifluoroacetic anhydride are added, during which the temperature rises to 40° C. The mixture is stirred at room temperature overnight. The mixture is then poured into 1 l of water and extracted three times with 0.5 l of ethyl acetate each time. The organic phase is washed with saturated sodium bicarbonate solution and with 1 N HCl, dried with MgSO4 and concentrated in a rotary evaporator.

Yield: 33.7 g (100% of theory) Melting point: 81° C. R_{f} (SiO₂, T1E1): 0.74

### IIIe) Methyl (2-fluorobenzyl)-1H-pyrazolo[3,4-b]pyridine-3-carboximidate

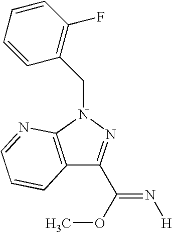

30.37 g (562 mmol) of sodium methoxide are dissolved in 1.5 l of methanol, and 36.45 g (144.5 mmol) of 3-cyano-1-(2-fluorobenzyl)-1H-pyrazolo[3,4-b]pyridine (from example IIId) are added. The solution obtained after stirring at room temperature for 2 hours is employed directly for the next stage.

### IIIf) 1-(2-Fluorobenzyl)-1H-pyrazolo[3,4-b]pyridine-3-carboxamidine

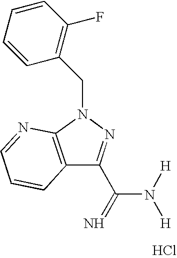

The solution of methyl (2-fluorobenzyl)-1H-pyrazolo[3,4-b]pyridine-3-carboximidate in methanol obtained from example IIIe) is mixed with 33.76 g (32.19 ml, 562 mmol) of glacial acetic acid and 9.28 g (173 mmol) of ammonium chloride and stirred under reflux overnight. The solvent is evaporated in vacuo, the residue is thoroughly triturated with acetone, and the precipitated solid is filtered off with suction.

¹H-NMR (d₆-DMSO, 200 MHz): δ=5.93 (s, 2H); 7.1-7:5 (m, 4 H); 7.55 (dd, 1H); 8.12 (dd, 1H); 8.30 (dd, 1H); 9.5 (bs, 4H-exchangeable) ppm. MS (EI): m/z=270.2 (M−HCl)

### EXAMPLES

### 1. 2-[1-(2-Fluorobenzyl)-1H-pyrazolo[3,4-b]pyridin-3-yl]-5-(8-oxa-3-azabicyclo[3.2.1oct-3-yl)-4-pyrimidinylamine

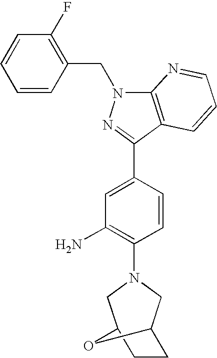

930 mg (3.33 mmol) of 1-(2-fluorobenzyl)-1H-pyrazolo[3,4-b]pyridin-3-carboximidamide from Ex. III and 1.00 g (4.50 mmol) of (E/Z)-2-cyano-2-(8-oxa-3-azabicyclo[3.2.1]oct-3-yl)ethenyl acetate from Ex. I, which had previously been freshly prepared, were suspended in 10 ml of toluene. The mixture was stirred at 120° C. overnight and then evaporated to dryness in a rotary evaporator. The residue was chromatographed in a preparative HPLC (method I). The product-containing fractions were subjected to a further purification by preparative HPLC (method I).

Yield: 230 mg (16%) R_{f} 0.23 (dichloromethane/methanol 20:1)

¹H NMR: (300 MHz, D₆-DMSO), δ=1.74-1.90 (m, 2H, CH₂), 2.07-2.20 (m, 2H, CH₂), 2.82-2.95 (m, 4H, 2CH₂), 4.29-4.42 (m, 2H, 2CH), 5.80 (s, 2H, CH₂), 6.40-6.70 (br, s, 2H, NH₂), 7.10-7.28 (m, 3H, Ar—H), 7.30-7.40 (m, 2H, Ar—H), 8.10 (s, 1H, pyrimidine H), 8.62 (dd, 1H, pyridine H), 8.97 (dd, 1H, pyridine H).

LC-MS: Rₜ=1.842 min (method E). MS (ESI pos.), m/z=432 ([M+H]⁺), 863 ([2M+H]⁺).

### 2. 2-[1-(2-Fluorobenzyl)-1H-pyrazolo[3,4-b]pyridin-3-yl]-5-(3-oxa-9-azabicyclo[3.3.1]non-9-yl)-4-pyrimidinylamine

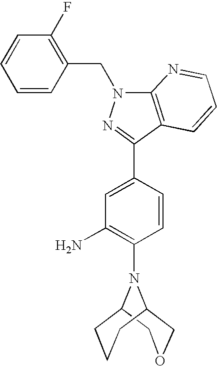

879 mg (3.14 mmol) of 1-(2-fluorobenzyl)-1H-pyrazolo[3,4-b]pyridin-3-carboximidamide from Ex. III and 1.00 g (4.23 mmol) of (E/Z)-2-cyano-2-(8-oxa-3-azabicyclo[3.2.1]oct-3-yl)ethenyl acetate from Ex. II, which had previously been freshly prepared, were suspended in 10 ml of toluene. The mixture was stirred at 120° C. overnight and then evaporated to dryness in a rotary evaporator. The residue was chromatographed in a preparative HPLC (method I). The product-containing fractions were subjected to a further purification by preparative HPLC (method I).

Yield: 141 mg (10%) R_{f} 0.24 (dichloromethane/methanol 20:1)

¹H NMR: (200 MHz, D₆-DMSO), δ=1.55-1.83 (m, 4H, 2CH₂), 1.97-2.20 (m, 2H, CH₂), 2.38-2.65 (m, 2H, CH₂), 3.80 (d, 2H, 2CH₂), 4.05 (d, 2H, 2CH₂), 5.87 (s, 2H, CH₂), 7.10-7.51 (m, 5H, Ar—H), 7.53-7.93 (br. s, 2H, NH₂), 7.88 (s, 1H, pyridine H), 8.72 (dd, 1H, pyridine H), 9.04 (dd, 1H, pyridine H).

LC-MS: Rₜ₌1.986 min (method E). MS (ESI pos.), m/z=446 ([M+H]⁺), 891 ([2M+H]⁺).

1. A compound of the formula (I) 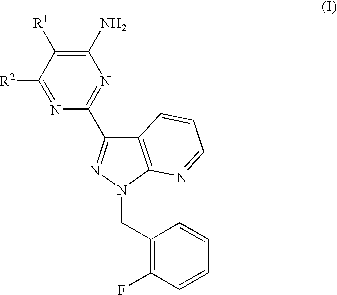
in which
R¹ is 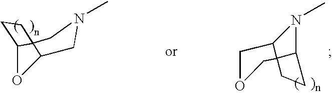
in which
n is 1 or 2;
R² is H or NH₂;
and salts, isomers and hydrates thereof:

2. A compound as claimed in claim 1,
in which
R¹ is 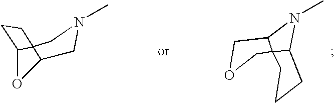
R² is H or NH₂;
and salts, isomers and hydrates thereof.

3. A compound as claimed in claim 1,
in which
R¹ is 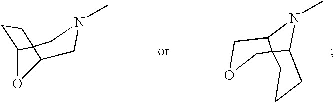
R² is H;
and salts, isomers and hydrates thereof.

4. A process for preparing the compound of the formula I, comprising the reaction of the compound of the formula (II) 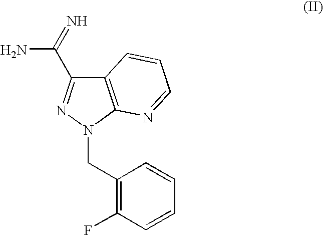
A) with a compound of the formula (III) 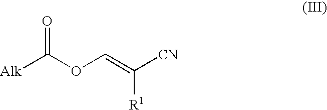
where
R¹ is as defined above;
Alk is linear or branched C₁₋₄-alkyl;
where appropriate in an organic solvent with heating to give the compound of the formula (I);
or
B) with a compound of the formula (IV) 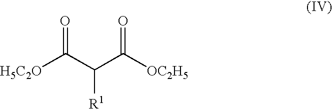
where
R¹ is as defined above;
in an organic solvent with heating to give compounds of the formula (V) 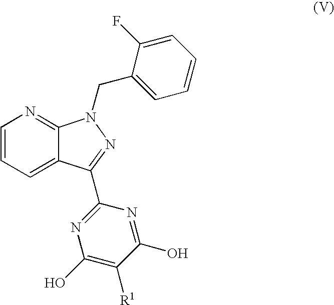
where
R¹ is as defined above;
subsequently with a halogenating agent to give compounds of the formula (VI) 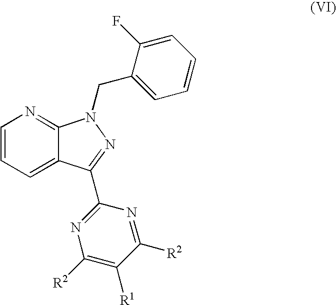
where
R¹ is as defined above;
R² is halogen;
and finally with aqueous ammonia solution with heating under elevated pressure.

5. A compound of the formula (I) for treating diseases.

6. A medicament comprising at least the compound of the formula (I) as claimed in claim 1.

7. A process for producing medicaments, characterized in that the compound of the formula (I) as claimed in claim 1 is converted into a suitable administration form, where appropriate with conventional excipients and additives.

8. A medicament comprising the compound of the formula (I) as claimed in claim 1 in combination with organic nitrates or NO donors.

9. A medicament comprising the compound of the formula (I) as claimed in claim 1 in combination with compounds which inhibit the breakdown of cyclic guanosine monophosphate (cGMP).

10. The use of the compound of the formula (I) as claimed in claim 1 for producing medicaments for the treatment of cardiovascular disorders.

11. The use of the compound of the formula (I) as claimed in claim 1 for producing medicaments for the treatment of hypertension.

12. The use of the compound of the formula (I) as claimed in claim 1 for producing medicaments for the treatment of thromboembolic disorders and ischemias.

13. The use of the compound of the formula (I) as claimed in claim 1 for producing medicaments for the treatment of sexual dysfunction.

14. The use of the compound of the formula (I) as claimed in claim 1 for producing medicaments having antiinflammatory properties.

15. The use of compounds of the general formula (I) as claimed in claim 1 for producing medicaments for the treatment of disorders of the central nervous system.

16. The use as claimed in any of claims 9 to 14, where the compound of the formula (I) as claimed in claim 1 is employed in combination with organic nitrates or NO donors or in combination with compounds which inhibit the breakdown of cyclic guanosine monophosphate (cGMP).
